(19) United States
(12) Patent Application Publication
(10) Pub. No.: US 2011/0313431 A1
(43) Pub. Date: Dec. 22, 2011

(54) SUTURE AND ANCHOR FOR PERIODONTAL PROCEDURES AND METHOD OF USING THE SAME

(75) Inventors: Daniel Andrew Shimko, Germantown, TN (US); Susan J. Drapeau, Cordova, TN (US)

(73) Assignee: WARSAW ORTHOPEDIC, INC., Warsaw, IN (US)

(21) Appl. No.: 12/818,353

(22) Filed: Jun. 18, 2010

(51) Int. Cl. A61B 17/10; A61B 17/04

(52) U.S. Cl. 606/139; 606/228; 606/232

(57) ABSTRACT

A suture system that includes at least one suture having an end portion, a loop portion configured to be disposed about a fixed point and an anchor configured to be connected to the end portion for anchoring the suture to tissue adjacent to the fixed point, wherein the loop portion of the suture is disposed about the fixed point so as to reduce a gap between the tissue and the fixed point. A kit including at least one suture system, a surgical tool for manipulating the suture and written instructions of use are disclosed. A method for using the suture system is also described.

### TECHNICAL FIELD

The present disclosure generally relates to medical devices for the treatment of periodontal disorders, and more particularly to sutures used in periodontal surgery.

### BACKGROUND

Any publications or references discussed herein are presented to describe the background of the invention and to provide additional detail regarding its practice. Nothing herein is to be construed as an admission that the inventors are not entitled to antedate such disclosure by virtue of prior invention.

Periodontal disease is commonly known as pyorrhea. It has been estimated that most adult tooth loss in the United States today is the result of periodontal disease, not dental caries (cavities). As periodontal disease progresses, the pockets become larger and are filled with bacteria and pus, and in time, destruction of the tissue attachment to the teeth and destruction of the supporting bone structure occurs. Periodontal disease is usually painless, site specific and goes through periods of exacerbation and remission. As the disease advances gingival sulcui, or gaps form between the gum tissue/bone and the tooth, enlarge and are often prone to infection.

It is common knowledge that early detection of this problem and periodic measurements at several locations, as many as six per tooth, is necessary to determine if the gingival sulcui are enlarging and how fast. If the sulcui enlarge to a degree that the oral surgeon believes that they need to be reduced or eliminated in order to preserve the future health of the teeth, oral surgery may be necessary. If oral surgery is necessary, sutures would most likely be used. In addition, good suture techniques in the oral cavity are a bit of an art, and often years of experience are required to perfect the art. Therefore, what is also needed is a suture that can be used to provide the perfect suture each time that is not dependent on the skill level of the surgeon doing the suturing. The present invention provides such sutures and is further described in the sections below.

### SUMMARY OF THE INVENTION

Accordingly, a dental suture system comprising a suture having an end portion and a loop portion wherein the loop portion is configured to be disposed about a tooth. The dental suture also comprises an anchor that is either connected to or connectable to the end portion of the suture. The anchor is configured to connect the suture to a tissue, for example to connect the suture to the gum tissue that is adjacent to one or more teeth in which the loop portion of the suture is disposed there about. This will allow tension to develop on the suture and draw the tissue adjacent to the tooth in which the loop is disposed so as to reduce the sulcui or gap formed between the gum and tooth.

In one embodiment of the invention, in accordance with the principles of the present disclosure, a dental suture system is provided comprising a suture having an end portion and a loop portion. The loop portion of the suture is configured to be disposed about a tooth. The dental suture system also comprises an anchor that is configured to be attached, or is in communication with the end portion of the suture and is designed to secure, anchor and/or connect the suture to a tissue adjacent to the tooth in which the loop portion of the suture is to be disposed there about. In other words, the anchor prevents the suture from pulling through the gum tissue when tension is applied to the suture as it is positioned around the adjacent tooth.

Also provided in accordance with the principles of the present disclosure is a method of reducing a gap between a tooth and an adjacent tissue comprising passing the suture described herein into tissue adjacent to the tooth in which the loop portion is to be disposed there about so that the anchor anchors/connects the suture to the tissue and positions the loop portion of the suture in such a position that allows the loop portion to be deposed about the tooth. The suture is secured, the loop portion of the suture is positioned about the tooth and tension on the suture is applied such that a gap between the tooth and the tissue is reduced. This can be repeated in the patient's mouth on several teeth as necessary.

In one embodiment of the invention, in accordance with the principles of the present disclosure an adjustable length dental suture/anchor system comprising an adjustable length suture is provided. The dental suture has a first end portion and a second end portion configured so that the first end portion and the second end portion can be attached together to form a loop portion. The loop portion configured to be disposed about a tooth. The dental system also comprises an anchor configured to receive the first and/or second end portion of the suture such that said loop portion is fixed to the anchor. The anchor is configured to connect the suture to a tissue adjacent to the tooth in which the loop portion is to be disposed so that tension is or can be created on the suture and a reduction of the gap size formed between the tooth the adjacent tissue occurs.

Similarly, in accordance with the principles of the present disclosure, a procedure using the adjustable length suture described above is provided. The method of reducing a gap between a tooth and an adjacent tissue comprises some or all of the following steps: determining the approximate length of the adjustable suture to fit about the tooth. Once the desired length is determined, the suture is threaded through or attached to the anchor to form a loop portion. Joining the first and second ends of the suture together to create the loop portion can do this. Once the loop portion is formed the suture is passed into tissue adjacent to the tooth so that the anchor of the suture attaches/secures the suture to the tissue and positions the loop portion of the suture towards the tooth in which the loop will be dispose. Once in position, the loop portion of the suture is placed about the tooth next to the tissue and tension is provided on the suture so that the gap between the tooth and the tissue is reduced. This can be repeated several times as necessary. For example, the suture may comprise an elastomeric material and/or the suture length may be adjusted once it is placed about the tooth so as to provide sufficient tension to pull the tissue towards the tooth.

In one embodiment of the present invention, a non-elastic suture configured so that it can be attached to the anchor at one end with the second end having a loop. The loop is configured so that it can be either looped over the anchor set in place once the suture is positioned over the tooth adjacent to the tissue where the anchor is located. The small loop also provides a gripping point for a needle that is used to guide thread the suture into the tissue adjacent to the tooth.

In yet another embodiment of the present invention, the anchor is configured to have a front plate and a locking back portion. The front plate of the anchor is configured to have at least one hole in which a free end of the suture can be passed through and either crimpled, tied or locked into place once the proper length of the suture is determined. In one embodiment of the present invention, the suture is configured to have a locking mechanism including an integrated gear rack and a ratchet within a small open case. That is, the suture is configured as a conventional cable tie and once one end of the cable tie is pulled through the case and past the ratchet, the suture is prevented from being pulled back. This configuration allows the suture to be tightened but prevents the suture from lossening. Once the suture is positioned over the tooth and secured in place, the back plate of the of the anchor can be snapped or screwed in place in order to further secure the suture to the anchor.

Still yet another embodiment of the present invention is directed to dental suture kits for reducing a gap between a tooth and an adjacent tissue. The kits comprise at least one adjustable and/or pre-sized suture with an anchor and, optionally, a surgical tool for cutting the adjustable suture to a desired length and/or manipulating the adjustable or pre-sized sutures into the tissue and/or around the tooth adjacent to the tissue so as to reduce the size of the gap between the tissue and the adjacent tooth. For example, the kit may comprise a suture needle, a hook configured to manipulate the loop and assist in placing the loop about the tooth and/or a tool configured to adjust the suture length and apply tension to the suture. The kits may include anyone of the suture/anchor systems described above. The kits may be sterilized and may be disposable.

These features, together with other objects and advantages which will become subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

The present disclosure will become more readily apparent from the specific description accompanied by the following drawings, in which:

FIG. 1 is a plan view, in part cross section, of one particular embodiment of a dental suture system in accordance with the principles of the present disclosure;

FIG. 2 is a plan view, in part cross section, of one particular embodiment of a dental suture system in accordance with the principles of the present disclosure;

FIG. 3 is a plan view, in part cross section, of a normal tooth and gum without a gap between the tooth and gum;

FIG. 4 is a plan view, in part cross section, of a diseased gum showing a gap between the tooth and gum;

FIG. 5A is a plan view, in part cross section, of a diseased gum showing a gap between the tooth and gum and a suture according to FIG. 1 being passed into the gum;

FIG. 5B is a plan view, in part cross section, of a diseased gum showing a gap between the tooth and gum and a suture according to FIG. 5A wherein the suture is passed into the gum and the anchor is in place;

FIG. 5C is a plan view, in part cross section, of a diseased gum showing a gap between the tooth and gum and a suture according to FIG. 5B wherein the suture is positioned over the tooth and the gap is reduced;

FIG. 6 is a plain view, in partial cross section, of a diseased gum showing a gap between the tooth and gum and a non-elastic suture having a free looped end and an the other end attached to the anchor;

FIG. 6A is a plain view, in partial cross section, of a diseased gum having the anchor inserted into the gum tissue and the suture positioned around the adjacent tooth;

FIG. 6B is a plain view of the suture in a circle with the loop being positioned over the anchor;

FIG. 6C is a plain view of the closed loop end of the suture and its design for tightening the loop for attachment to the anchor;

FIG. 7 is a plain view of a suture/anchor system of the present invention wherein the anchor has a pass through hole in a faceplate and an attachable back plate;

FIG. 7A is a plain view of the suture/anchor system of FIG. 7 with the suture feed through the pass through hole of the faceplate of the anchor;

FIG. 7B is a plain view of the suture/anchor system showing a back plate configured to fit into anchor;

FIG. 8 is a plain view of a suture configured to have a ratchet and locking mechanism;

FIG. 9A is a plain view of a one needle that can be used I the present invention; and

FIG. 9B is a plain view of a one needle that can be used I the present invention.

Like reference numerals indicate similar parts throughout the figures.

### DETAILED DESCRIPTION OF THE INVENTION

The exemplary embodiments illustrated in the Figures show sutures of the present invention that can be used to reduce or eliminate a gap or suclui between a tooth and adjacent gum as a result of periodontal disease or following dental surgery. As periodontal disease advances, the gaps typically increase in size causing food particles to accumulate therein, which then facilitates bacterial growth that may eventually lead to tooth decay. Normal brushing typically does not fully reach all of the area in the gaps, eventually leading to additional gum disease, tooth decay and eventually tooth loss.

One procedure that can be used to reduce or eliminate the gaps that form in periodontal disease is gum grafting. That is, taking gum tissue from one portion of the mouth, usually the roof of the mouth, and grafting it to the gum tissue where the gap is formed in order to close or eliminate the gap. However, this procedure is painful, costly and does not guarantee that the gap will not form again. The suture of the present invention can be used to reduce or eliminate the gap without grafting gum tissue. The suture can also be replaced or adjusted to further reduce the gap should it re-develop or as it is reduced in size. In addition, the present invention can be used after standard conventional periodontal procedures such as “scaling and root planning” have been completed.

In many instances, a patient with a periodontal defect (which typically involves the root surface, adjacent bone, and the connective periodontal tissue) adjacent to a tooth is placed under local anesthesia and surgical instruments (elevators and scalpels) are used to reflect the gum tissue overlying and adjacent to the diseased area. The diseased tissues of the periodontal defect are debridded, typically by hand scraper, ultrasonic debridders, etc., and the root surface/defect may or may not received a biologic agent, chemical agent, or a bone-grafting material (whose may serve either to foster regrowth of the lost anatomic structures or to encourage re-attachment of the soft tissue to the tooth following wound closure). The gum tissue is then repositioned to its appropriate anatomic location and sutured in place. Appropriate repositioning of this gum tissue and reduction of the sulcui are key to success as this tissue acts as the primary barrier to infectious agents that may re-enter the wound that was just so carefully treated.

The present invention may be understood more readily by reference to the following detailed description of the invention taken in connection with the accompanying figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

While the invention is described in the context of gum disease and encouraging gum tissue towards a tooth, that is simply to assist the reader in understanding one particular use and in light of the specification it will be clear that the invention can be used to encourage any soft tissue in the direction of the loop attachment, be that a tooth, an implant or any other suitable structure.

Also, as used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

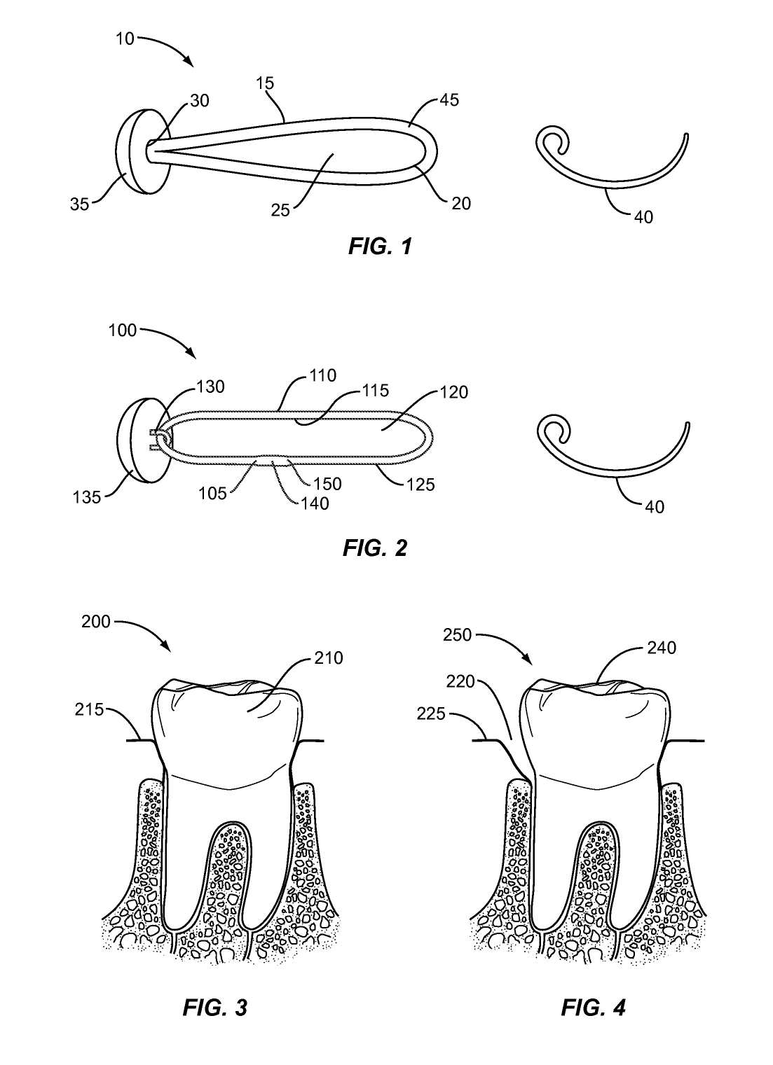

The following discussion includes a description of a dental suture, related components and exemplary methods of employing the dental suture in accordance with the principles of the present disclosure. Alternate embodiments are also disclosed. Reference will now be made in detail to the exemplary embodiments of the present disclosure, which are illustrated in the accompanying figures. Turning now to FIGS. 1-9, there are illustrated components of the dental suture system in accordance with the principles of the present disclosure.

FIG. 1 shows a plan view, in part cross section, of one particular embodiment of a dental suture system in accordance with the principles of the present disclosure. As shown, the suture system (10) comprises an anchor (35) and a suture (45) attached to the suture (45) at an attachment point (30). The suture (45) has an outside surface (15) and an inside surface (20) and is configured so as to be attached to anchor (35) at attachment point (30). The suture (45) is configured as a loop with the inside surface (20) of the suture (45) creating a lumen (25) configured to fit around a tooth adjacent to tissue in which it is used. The suture system (10) may also include a needle (40) designed to have a pointed sharp end and a suture attachment end, wherein the suture attachment end has a suture attachment mechanism such as a port that allows the suture to be removably secured to the suture needle. The suture attachment end is configured to hold the suture (45) while the pointed end is configured to guide the suture (45) into and/or through the soft tissue so that the anchor (35) secures the suture to the tissue in a configuration where the suture (45) can be stretched over an adjacent tooth. With tension on suture (45), the gum is pulled or encouraged towards the tooth so that the gap between the tooth and the tissue is reduced. In this exemplary embodiment, the suture is of a predetermined size and the size cannot be adjusted. The suture may be made of any suitable material, including an elastomeric material.

FIG. 2 shows a plan view of another embodiment in accordance with the principles of the present disclosure. In this embodiment, the suture system (100) comprises an anchor (135) having a suture-anchoring member (130) configured to accept a suture (125) threaded through the anchoring member (130) so as to secure the suture (125) in a tissue. The suture (125) has a first end (105) and a second end (150) and is configured so that it can be cut to size. Once it is cut to size the suture (125) can be threaded through the anchor member (130) and the first end (105) and the second end (150) affixed to one another at point (140). This produces a suture (125) that is configured as a loop with an inside surface (115) and an outside surface (110) wherein the inside surface (115) outlines a lumen (120) that is configured to fit around a tooth adjacent to tissue in which it is used. Since the suture (125) can be cut to size prior to or after attaching it to the anchor (135), the suture (125) may be custom designed to fit the particular tooth and application in which is to be used. This allows the suture (125) to be modified to fit as tight as desired so as to be productive in reducing or elimination the gap between the gum and tissue.

As with the suture system shown in FIG. 1, the suture system (100) shown in FIG. 2 may also include a needle (40) designed to have pointed sharp end and a curled end that is able to grab the suture (125) and pass it into soft tissue so that the anchor (135) secures the suture (125) to the tissue in a configuration where the suture (125) can be stretched over the adjacent tooth to reduce or close the gap between the tooth and the tissue. Various shaped needles (40) can be used to thread the suture (125) into the adjacent tissue and into position around the adjacent tooth. For example, a needle (40) used in the present invention can have a semi-circular shape with one pointed end for threading the suture into tissue and one end configured to capture the suture (125) so that it can be easily released once the suture (125) is passed through the tissue. The suture capturing end can have a hole to pass the suture (125) to be threaded into a tissue in or can have a claw type shape to grab the suture (125) and hold the suture (125) as it is passed into the tissue. Once threaded into the tissue the suture (125) can be undone from the claw or hole so as to easily release the suture (125) without disturbing the positioning of the suture (125) around the tooth. Exemplary needle configurations are shown in FIGS. 9A and 9b.

FIG. 3 and FIG. 4 show plan views, in part cross section, of a normal tooth and gum (200) without a gap between the tooth and gum (FIG. 3) and a diseased gum having a gap between the gum and tooth (250) (FIG. 4). With reference to FIG. 3, the gum (215) is shown to be in close proximity to the tooth (210) so that no gap is present between the tooth (210) and the gum (215). FIG. 4, on the other hand, shows a gum (225) and tooth (240) arrangement having a gap (220). The diseased gum/tooth arrangement (250) shown in this figure is the type of problem that the suture system (10, 100) of the present invention is designed to correct. As described herein, either predetermined suture size systems (10), such as shown in FIG. 1 can be used or an adjustable length suture system (100), such as shown in FIG. 2 can be used.

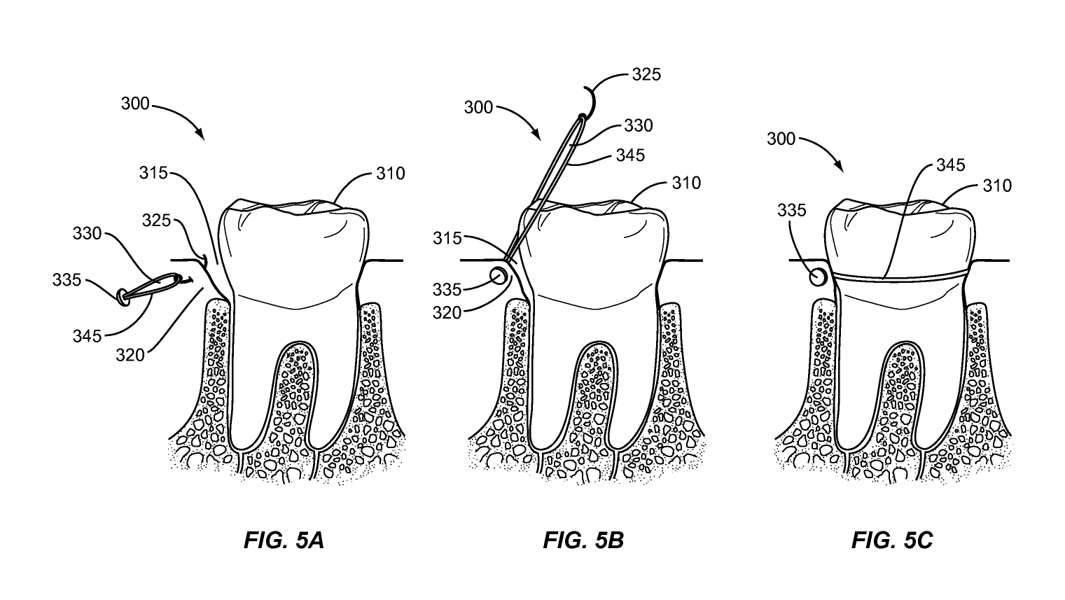

FIGS. 5A-5C show a plan view, in part cross section, of a diseased gum having a gap between the tooth (310) and gum tissue (320) which illustrates the procedure (300) of passing the suture (345) through the gum tissue (320) and lassoing the adjacent tooth (315) in order to reduce or eliminate gap (315). Suture (345) is passed through gum tissue (320) using needle (325) at a position next to gap (315) to be closed. FIG. 5B shows the suture (345) passed through the gum tissue (320) so that anchor (335) is anchoring the suture (345) to the gum tissue (320). In this position, the lumen (330) of the suture (345) is in close proximity to the tooth (310) so that it can be positioned over the adjacent tooth (310). The progression from FIG. 5B to 5C shows the lassoing of adjacent tooth (310) by the suture (345), which produces the effect of closing the gap (315) between the gum (320) and the tooth (310). In FIG. 5C, the anchor (335) is positioned flat against gum tissue (320) so as to not affect the recipient and suture (345) is lassoed around tooth (310) and preferably is positioned so as to not affect the recipient when chewing. This can be repeated on adjacent teeth until all of the gaps are reduced or eliminated.

Since the suture (345) constitutes only a minor intrusion of the gum tissue (320), should the gum tissue (320) adhere or stretch to naturally fill the gap and there is no further need for the suture system, the suture and/or anchor (335) can be removed without major surgery. Similarly, should the suture (345) brake or stretch and the gap re-appear, the old suture (345) can be removed and replaced with a new suture (345) and/or anchor (335) of the same or a different length. In the case where the gap (315) is large multiple sutures (345) may be used in order to close or eliminate the gap (315).

The components of the anchor portions of the suture system, may be fabricated from materials suitable for medical applications, including metals, synthetic polymers, ceramics, bone, bio-compatible materials, commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate ceramics (e.g. SKELITE™), rigid polymers including polyphenylene, polyamide, polyimide, polyetherimide, polyethylene, polyurethanes of any durometer, epoxy and silicone, manmade materials and/or their composites. What material the anchor is constructed of depends on the particular application and/or preference of a medical practitioner. The anchor may also have a relatively large surface area (or possess integrated features (i.e. hooks, barbs, surface texture, porosity, etc) that limit pull-though) that aids in retaining the suture/anchor in the soft tissue. The size of the anchor depends directly on the tension that is needed to encourage the soft tissue over to the tooth in order to close the gap between the tooth and the gum. In general, the more tension that is necessary to move the gum towards the tooth to close the gap the greater the necessary surface area of the anchor.

The suture of the suture system may be mono or multi-filament and fabricated from materials suitable for medical applications, including Polyglycolic acid, Polyester—braided and unbraided, polyester fiber, T-CRON, Silk-unbraided, Silk-braided, PERMA-HAND silk, black silk, surgical silk, Braided coated silk, Gut, Chromic gut, Nylon, PGA sutures, Polybutester sutures, Polyglycolic sutures, Polypropylene sutures, PTFE sutures, Monomid black nylon, NUROLON braided nylon suture, Dermapoint nylon, Dermalon, monofilament, manmade materials as well as other materials suitable for dental suture materials and mixtures thereof. The adjustable sutures of the present invention can be equipped with sticky ends for adhering together or can be integrally connected, fastened and/or tied.

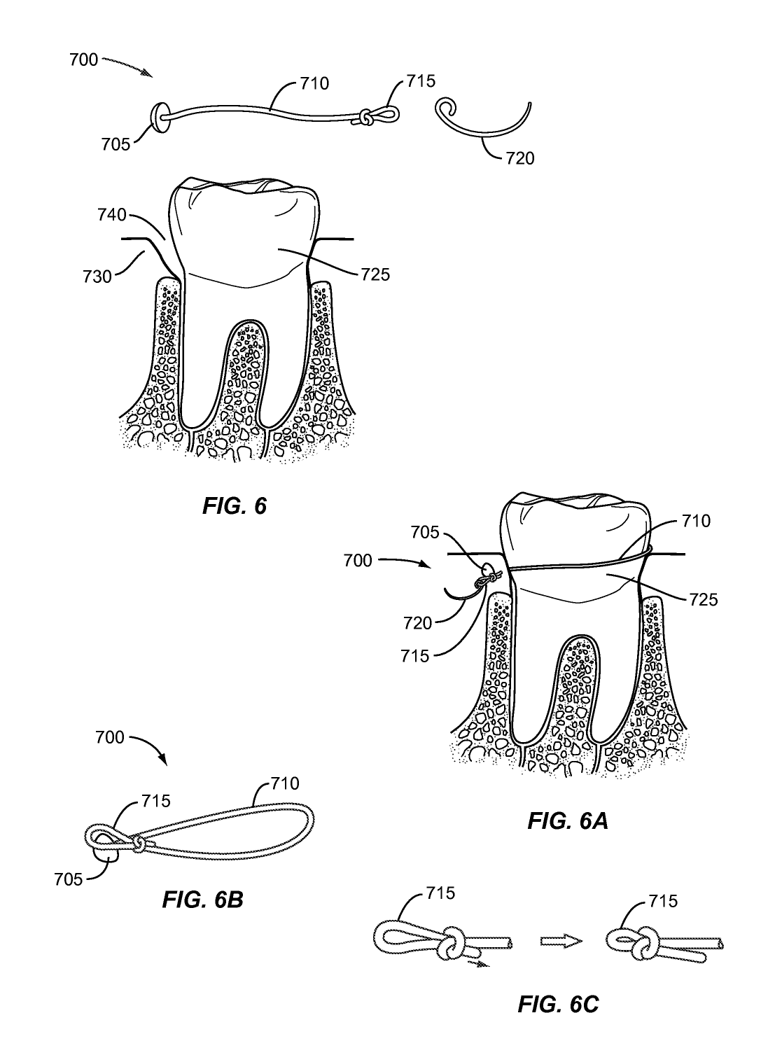

FIG. 6 shows one embodiment of the suture/anchor system of the present invention (700). The suture (710) is configured to have an anchor (705) at one end and a loop (715) at the other. The loop (715) is used as a grasping point for the suture (710) by the needle (720). Once the needle (720) grasps the suture (710), the needle (720) is used to thread the suture (710) into tissue (730) adjacent to tooth (725). Once tension is placed on the suture (710), the gap (740) formed between the tooth (725) and the adjacent tissue (730) is closed. FIG. 6A shows the needle (720) threaded through tissue (730) and suture (710) positioned around tooth (725). FIG. 6B shows the positioning of the suture (710) around the anchor (705). In order to adjust tension on the suture (710), as shown in FIG. 6C, the free end of the suture can be pulled tight over anchor (705) in order to reduce the chance of slipping off of the anchor (705). A slip knot or other similar mechanism can be used to ensure tension remains on the suture.

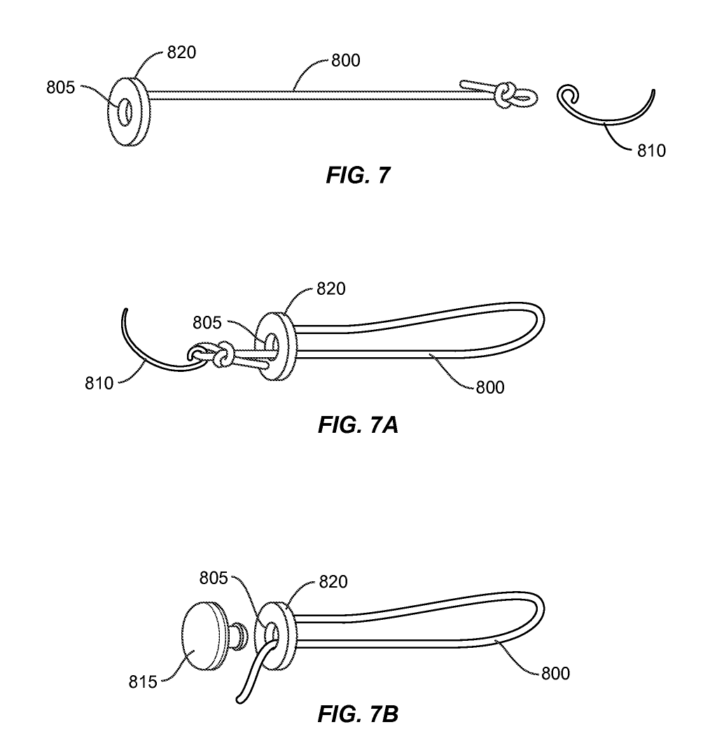

FIGS. 7, 7A and 7B show an embodiment of the present invention where the suture (800) is passed through a passageway (805) in the anchor (820) to form a loop. The suture (800) is then positioned about the tooth and tightened to exert tension on the suture (800). Once tension is applied the suture (800) is tied off using any of the mechanisms described herein, excess suture (800) material may be trimmed and discarded and anchor back plate (815) is either snapped or screwed in place (e.g., into the passageway (805)) to assure that the suture (800) remains taught. Tension on the suture (800) may be adjusted from time to time by undoing the anchor back plate (815) and pulling the suture (800) tighter, then replacing the anchor back plate (815) on the anchor (820).

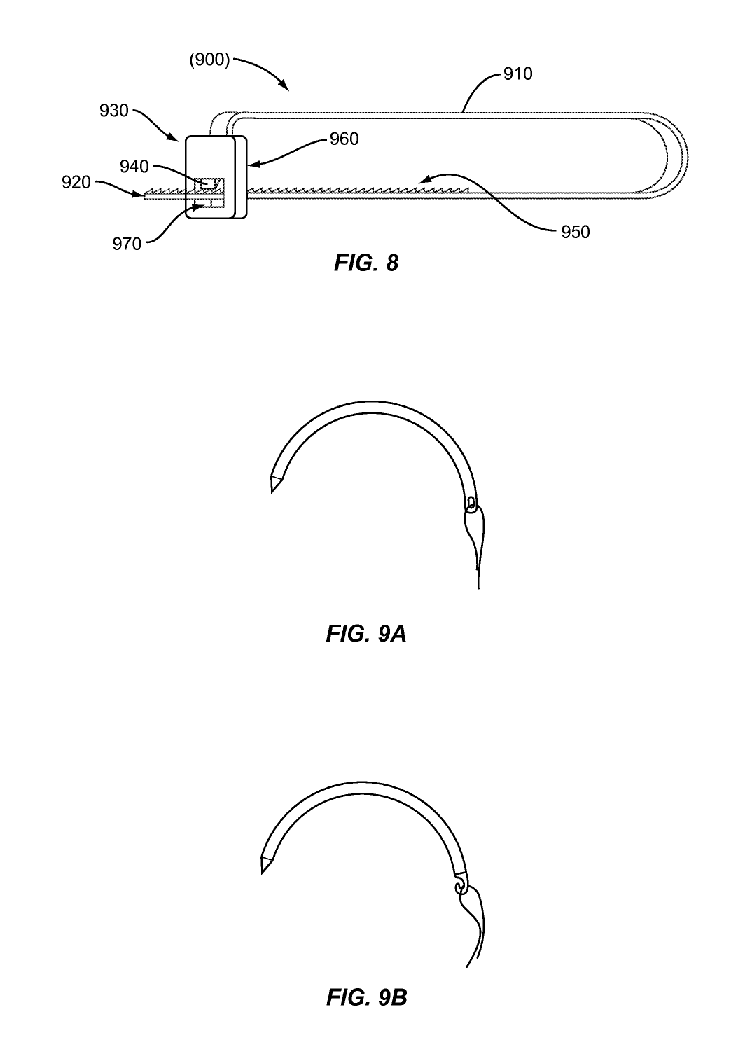

Another exemplary embodiment of the present invention (900) is shown in FIG. 8. Here, the suture (910) is configured as a conventional cable tie and once a lead end (920) of the cable tie is pulled through a case and/or anchoring system (930) and past a pawl (940), the suture (910) is prevented from being pulled back by a ratchet or tooth system (950) on the suture (910). This configuration allows the suture to be tightened but prevents the suture (910) from loosening. Once the suture (910) is positioned over a tooth and secured in place, for example, a back plate (shown as 815 in FIG. 7B) of the anchor could be configured with a pawl (940) system in passageway (805) of FIG. 7B. Alternatively, the anchoring system (930) may comprise an anchor retraining surface (960) with the pawl (940) contained in a suture fastening passage (970) within the anchoring system (930).

The suture, as well as the anchor, can be coated, impregnated with or can contain agents to stop advancement of periodontal disease, relieve the pain associated with periodontal disease, and/or stimulate gum growth/healing to reverse effects of periodontal disease. Agents that can be used include flavorings, antibiotics, pain relieve medication, anti-inflammatory agents, antiseptics, tissue growth factors, bone growth factors, vitamins, tissue healing agents, plaque and tartar removing agents, plaque and tartar retardants and combinations thereof. These agents can be made to release over time in the vicinity of the suture or released rapidly once when put in place.

Another embodiment of the present invention is a kit containing a plurality of sutures and/or anchors that can be of the same size or various sizes. The kit may also include a suture that may be adjusted in length at the time of, or following, implanting of the suture system, as well as the pre-sized sutures, wherein the suture may be elastomeric and may be coated with one or more agents. The kit may also include agents and/or solutions to soak the sutures in prior to deployment. The kit may also include sutures made from different materials and colors so as to meet different situations that may arise. The kit may also provide dental utensils for positioning anchor and/or the suture system so as to be most effective.

The present invention is also directed to a method of reducing a gap between a tooth and an adjacent tissue comprising passing the suture of the present invention into tissue adjacent to the tooth in which the loop portion is to be disposed. Once the suture is passed into the gum tissue, the anchor is pulled tight against the tissue in which it is inserted. Once in place the suture is positioned so that the loop portion of the suture can be deployed about the adjacent tooth. Stretching and/or positioning the loop portion of the suture about the tooth thereby providing tension on the suture so as to reduce or eliminate the gap between the tooth and the tissue in which it is inserted.

When an adjustable suture length is used, the distance between the anchor point and the tooth may be measured or estimated so that an appropriate length of suture material can be determined. Once the length of the suture is determined the suture is cut to the desired size. The cut suture may then be attached to the anchor and may then be tied together to create a loop portion that can then be placed around an adjacent tooth. Alternatively, the suture may be passed around the tooth and connected to the anchor and then adjusted to create the desired amount of tension and fixed in length, for example, by forming a knot using the two ends of the suture.

In accordance with the principals of the present invention, the suture system of the present invention can be individually sterilized and packaged separately or as part of a kit. In the alternative, the entire kit can be sterilized and once it is opened the remaining sutures and/or anchors disposed of.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A medical suture system comprising;
a suture having an end portion and a loop portion, said loop portion configured to be disposed about a fixed attachment point; and
an anchor in communication with said end portion of said suture, said anchor configured to connect said suture to a tissue adjacent to said fixed attachment point on which said loop portion is to be disposed there about.

2. The medical suture system according to claim 1, wherein said suture is configured to be passed through said soft tissue adjacent to said fixed attachment point in which said loop portion of said suture is to be disposed, said anchor in communication with said suture being configured to anchor said suture in said tissue such that disposing said loop portion of said suture about said fixed attachment point creates tension on said suture and urges said soft tissue toward said fixed attachment point.

3. The medical suture system according to claim 1, wherein said anchor is fixed to said suture and said suture has a predetermined length.

4. The medical suture system according to claim 2, wherein said suture is made from an elastomeric material.

5. The medical suture system according to claim 1, wherein the suture comprises a ratchet and the anchor comprises a passageway having a pawl configured to engage the ratchet on the suture.

6. The medical suture system of claim 1, wherein the anchor has a first surface having an area substantially larger than said suture, a second surface and at least one sidewall disposed between said first surface and said second surface, wherein said loop portion of said suture is affixed to and extends from said first surface.

7. The medical suture system of claim 1, wherein the fixed point is a tooth or a medical implant.

8. An adjustable length suture/anchor system comprising;
an adjustable length suture having a first end portion and a second end portion, said first end portion and said second end portion are adapted to form a loop portion upon joining, said loop portion configured to be disposed about a fixed point;
an anchor configured to received said first and/or second end portion of said suture prior to said adjustable length suture being joined such that said loop portion is fixed to said anchor, said anchor configured to connect said suture to a tissue adjacent to said fixed point in which said loop portion is to be disposed there about so that tension created on said suture reduces the size of a gap formed between said fixed point and said adjacent tissue.

9. The suture system according to claim 8, wherein said adjustable suture is made from materials selected from group consisting of elastomeric material, monofilament, Polyglycolic acid, Polyester—braided and unbraided, polyester fiber, T-CRON, Silk-unbraided, Silk-braided, coated silk, Gut, Chromic gut, Nylon, Polybutester, Polyglycolic, Polypropylene, Monomid black nylon and mixtures thereof.

10. The suture system of claim 9, wherein the anchor has a first surface having an area substantially larger than said adjustable length suture, a second surface and at least one sidewall disposed between said first surface and said second surface and wherein said loop portion of said adjustable length suture extends from said first surface.

11. The suture system of claim 10, wherein the anchor comprises at least one passageway configured to allow slidable passage of at least one end of said adjustable length suture therethrough.

12. The suture system of claim 11, wherein the anchor comprises at least two passageways, each configured to allow slidable passage of an end of said adjustable length suture.

13. The suture system of claim 12, wherein the at least one passageway comprises an aperture having a pawl and the adjustable length suture comprises ratchet serrations on at least one side, wherein the pawl and aperture are configured to engage the ratchet serrations of the adjustable length suture when a free end of the adjustable length suture is passed through the aperture.

14. A method of reducing a gap between a fixed point and an adjacent soft tissue comprising;
(a) passing a suture through tissue adjacent to a fixed point in which said loop portion is to be disposed there about so that an anchor connected to said suture and that attaches said suture to said tissue and positions said loop portion of said suture such that said loop can be deposed about said tooth; and
(b) positioning said loop portion of said suture about said tooth thereby providing tension on said suture such that a gap between said tooth and said tissue is reduced.

15. The method according to claim 14, wherein the fixed point is a tooth or a medical implant.

16. The method according to claim 15, wherein said suture comprises a suture having a first and a second end portions and the method further comprising:
(a) cutting said suture to size;
(b) attaching said cut suture to said anchor; and
(c) joining said first and said second end portions together to create said loop portion.

17. The method according to claim 16, further comprising:
adjusting the length of the loop portion so as to apply a desired amount of tension to the anchor; and
encouraging the tissue to a position closer to the tooth or medical implant.

18. The method according to claim 15, wherein said suture comprises a suture having a first end portion attached to said anchor and a second end portion free from said anchor, and wherein said anchor comprises a passageway configured to receive and secure said second end portion of said suture, the method further comprising;
passing said second end portion of said suture through tissue adjacent to a fixed point;
inserting said second end portion of said suture through said passageway; and
securing said second end portion of said suture in said passageway, thereby preventing said suture from exiting said passageway.

19. A surgical kit for reducing a gap between a fixed point and adjacent tissue, the kit comprising;
an anchor in communication with a suture, wherein the anchor comprises a first surface having an area substantially larger than the cross-sectional area of the suture, a second surface and at least one sidewall disposed between said first surface and said second surface and wherein the suture comprises a loop portion extending from said first surface;
a surgical tool for manipulating said suture into said tissue; and
instructions on how to use said surgical kit.

20. The kit of claim 19, further comprising a plurality of sutures of variable sizes and wherein said sutures a made of an elastomeric material.
